(19) United States
(12) Patent Application Publication
(10) Pub. No.: US 2013/0316351 A1
(43) Pub. Date: Nov. 28, 2013

(54) METHODS AND KITS FOR DIAGNOSING CONDITIONS RELATED TO HYPOXIA

(71) Applicant: Osnat Ashur-Fabian, Zur Moshe (IL)

(72) Inventor: Osnat Ashur-Fabian, Zur Moshe (IL)

(21) Appl. No.: 13/702,851

(22) PCT Filed: Jun. 6, 2011
(86) PCT No.: PCT/IL2011/000444

§ 371 Date: Feb. 28, 2013

Related U.S. Application Data
(60) Provisional application No. 61/352,019, filed on Jun. 7, 2010.

(51) Int. Cl. C12Q 1/68

(52) U.S. Cl. CPC C12Q 1/6886; C12Q 1/6883
USPC 435/6.12

(57) ABSTRACT

The present invention provides a method for detecting a condition associated with hypoxia in a subject, a method for determining the severity of a condition associated with hypoxia, a method for determining the effectiveness of a therapeutic treatment of a condition associated with hypoxia and a method for selecting a subject suffering from a condition associated with hypoxia, to receive therapeutic treatment, wherein the methods of the invention are based on measuring the level of a cell free Ribonucleic acid (RNA) of a p53 inducible gene in the subject. The present invention is also directed to kits for performing the method of the invention.

### FIELD OF THE INVENTION

The invention relates to methods for detecting conditions associated with hypoxia in particular cerebrovascular accident, fetal stress and cardiovascular diseases.

### BACKGROUND OF THE INVENTION

Tissue hypoxia is a pathological condition in which tissue cells are deprived of adequate oxygen supply. When this occurs, normal biological processes in the cell are compromised in order to metabolically adapt to the oxygen deficiency. Oxygen-deprivation leads to the up-regulation of genes associated with numerous processes, such as vascularization and glycolysis, including erythropoietin and vascular endothelial growth factor.

Ischemia is defined as inadequate blood supply (circulation) to a local area due to blockage of the blood vessels to the area. This, in turn, leads to tissue hypoxia or anoxia (absence of oxygen). Ischemia always results in hypoxia; however, hypoxia can occur without ischemia if, for example, the oxygen content of the arterial blood decreases as occurs with anemia. Ischemic heart disease (IHD), or myocardial ischemia, is a disease characterized by ischaemia to the heart muscle, usually due to coronary artery disease (atherosclerosis of the coronary arteries). An estimated 14 million people in the United States have ischemic heart disease. Of these, as many as 4 million have few or no symptoms and are unaware that they are at risk for angina (angina pectoris) or heart attack (myocardial infarction).

p53 (also known as protein 53 or tumor protein 53), is a tumor suppressor protein that in humans is encoded by the TP53 gene (Matlashewski G, ET AL. Embo J. (1984), 3(13): 3257-62). p53 is important in multicellular organisms, where it regulates the cell cycle and thus functions as a tumor suppressor that is involved in preventing cancer. The activation of the p53 gene results in the transcriptional elevation of many target genes, including Apaf-1 and p21, some of which by 30 to 50 fold (Kannan et al., Oncogene (2001) 20(26):3449-55; Kannan et al., Oncogene, 2001; 20(18):2225-34).

Wild-type p53 protein is referred to as having a guardian-like role because it is responsible for monitoring the cellular state and responding to stress by inducing either a cell cycle arrest or apoptosis. Recent data shows that, although hypoxia-induced p53 does not transactivate known target genes, such as Apaf-1 or Perp, it binds to the promoters of these genes (Sumiyoshi Y, et al. Clin Cancer Res, 2006; 12: 5112-7). Other studies show that cellular levels of p53 are stabilized during hypoxia (Hammond E M et al. Clin Cancer Res (2006) 12(17):5007-5009).

Previous work showed that placentas of pregnancies complicated by fetal growth retardation, such as preeclampsia, exhibit enhanced apoptosis and up-regulation of p53 compared to normal pregnancies (Levy et al., Am J Obstet Gynecol (2002) 186(5):1056-61).

Ferguson-Smith has documented that small numbers of nucleated fetal cells, such as fetal trophoblasts, as well as cell-free fetal DNA pass into the maternal circulation. In preeclampsia the number of nucleated fetal cells and cell-free fetal DNA are increased, the latter even before clinical signs are apparent. Fetal DNA levels increase during pregnancy and are cleared in a matter of hours after delivery (Ferguson-Smith, Proc Nati Acad Sci USA, 2003; 100(8):4360-2). Similarly, fetal DNA can be detected in small amounts in the maternal circulation (Lo Y M, Corbetta N, Chamberlain P F, Rai V, Sargent I L, Redman C W, et al. Lancet, 1997; 350: 485-7). Thus, circulating nucleic acids can be found in the plasma and serum. The nucleic acids can be RNA, mitochondrial DNA or genomic DNA. Both DNA (at 1.8-35 ng mL-1) and RNA (2.5 ng mL-1) are found in the plasma and serum of healthy individuals and their levels rise in patients with various cancers, trauma, myocardial infarction and stroke.

Poon and colleagues have demonstrated for the first time, using a two-step reverse transcriptase (RT)-PCR assay, the presence of fetal-derived, male specific mRNA in plasma of pregnant women carrying male fetuses providing a means of noninvasive prenatal diagnosis (Poon et al., Clin Chem, 2000; 46(11):1832-4).

Circulating nucleic acids have also been shown to be useful as prognostic and predictive markers in patients with solid neoplasias (Goebel G, Dis Markers, 2005; 21(3):105-20). Cheng T, et al, reported that circulating c-met is an independent negative prognostic indicator in non-small cell lung cancer (Chest, 2005; 128: 1453-60).

Measurement of plasma circulating mRNA has been reported to enable early detection of hepatic injury (Kudo Y, et al., J Vet Med Sci, 2008; 70: 993-5).

Earlier studies concerning the existence of circulating nucleic acids in plasma and serum were mainly directed to the field of fetal medicine and oncology.

To date, circulating nucleic acids related studies involve other pathological states including trauma, sepsis, myocardial infarction, stroke, transplantation, diabetes mellitus and hematologic disorders (Butt and Swaminathan, Ann N Y Acad Sci. 2008; 1137:236-42). By introducing the highly sensitive one-step real-time quantitative reverse-transcription (RT)-polymerase chain reaction (PCR), circulating free RNA, which often only exists at low concentrations in plasma and serum, can be readily detected and quantified (Nancy B. Y, et al. Methods In Molecular Biology, 2006 Volume 336; pp: 123-134).

Corrias M V, et al, reported on the detection of cell-free RNA in children with neuroblastoma (NB) and comparison with that of whole blood cell RNA and suggested that for monitoring disease status detection of cell-free tumor-specific RNAs in patients with NB is not a reliable alternative to whole cell RNA. (Pediatr Blood Cancer, 2010; 54:897-903).

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention:

FIG. 1 is a graph showing p21 gene levels in normal versus hypoxic pregnancies. Results are depicted in triplicates and normalized to beta-actin level for each sample.

FIG. 2 is a graph showing p21 levels in normal (N) versus hypoxic pregnancies (H). Results are depicted in triplicates and normalized to beta-actin level in each sample.

### SUMMARY OF THE INVENTION

In one of its aspects the present invention provides a method for detecting a condition associated with hypoxia in a subject, the method comprising determining in a biological sample obtained from the subject the level of a cell free Ribonucleic acid (RNA) of at least one p53 inducible gene, wherein a level of the cell free RNA above or below a predetermined range associated with the at least one p53 inducible gene, is indicative that the subject has a condition associated with hypoxia.

In another one of its aspects the present invention provides a method for determining the severity of a condition associated with hypoxia in a subject comprising determining the level of a cell free RNA of at least one p53 inducible gene in a biological sample obtained from the subject and comparing the level of the cell free RNA of the p53 inducible gene with a predetermined range that correlates the level of the at least one p53 inducible gene with the severity of the condition associated with hypoxia, the comparison allowing determination of the severity of the condition associated with hypoxia in the subject.

In still another one of its aspects the present invention provides a method for determining the effectiveness of a therapeutic treatment of a condition associated with hypoxia in a subject comprising determining the level of cell free RNA of at least one p53 inducible gene in two or more biological samples obtained from the subject at two or more time points, at least one of the time points being during or after the treatment, wherein:
(i) for a p53 inducible gene that is over-expressed in a condition associated with hypoxia a decrease in the level of the cell free RNA of the p53 inducible gene between the two or more samples being indicative of effectiveness of the therapeutic treatment;
(ii) for a p53 inducible gene that is repressed in a condition associated with hypoxia an increase in the level of the cell free RNA of the p53 inducible gene between the two or more samples being indicative of effectiveness of the therapeutic treatment.

In still yet another one of its aspects the present invention provides a method for selecting a subject suffering from a condition associated with hypoxia, to receive therapeutic treatment to treat the condition, the method comprising determining the level of cell free RNA of at least one p53 inducible gene in a biological sample obtained from the subject and selecting the subject to receive said therapeutic treatment if the level of cell free RNA of at least one p53 inducible gene is above or below a predetermined range associated with the at least one p53 inducible gene.

### LIST OF EMBODIMENTS

Disclosed below are some non-limiting embodiments of the invention, provided in the form of numbered paragraphs. A method for detecting a condition associated with hypoxia in a subject, the method comprising determining in a biological sample obtained from the subject the level of a cell free Ribonucleic acid (RNA) of at least one p53 inducible gene, wherein a level of the cell free RNA above or below a predetermined range associated with the at least one p53 inducible gene, is indicative that the subject has a condition associated with hypoxia.
1. A method for detecting a condition associated with hypoxia in a subject, the method comprising determining in a biological sample obtained from the subject the level of a cell free Ribonucleic acid (RNA) of at least one p53 inducible gene, wherein a level of the cell free RNA above or below a predetermined range associated with the at least one p53 inducible gene, is indicative that the subject has a condition associated with hypoxia
2. A method for determining the severity of a condition associated with hypoxia in a subject comprising determining the level of a cell free RNA of at least one p53 inducible gene in a biological sample obtained from the subject and comparing the level of the cell free RNA of the p53 inducible gene with a predetermined range that correlates the level of the at least one p53 inducible gene with the severity of the condition associated with hypoxia, the comparison allowing determination of the severity of the condition associated with hypoxia in the subject.
3. A method for determining the effectiveness of a therapeutic treatment of a condition associated with hypoxia in a subject comprising determining the level of cell free RNA of at least one p53 inducible gene from two or more biological samples obtained from the subject at two or more time points, at least one of the time points is during or after the treatment, wherein:
   (i) for a p53 inducible gene that is over-expressed in a condition associated with hypoxia a decrease in the level of the cell free RNA of the p53 inducible gene between the two or more samples being indicative of effectiveness of the therapeutic treatment;
   (ii) for a p53 inducible gene that is repressed in a condition associated with hypoxia an increase in the level of the cell free RNA of the p53 inducible gene between the two or more samples being indicative of effectiveness of the therapeutic treatment.
4. The method of Embodiment 3, wherein one or more first samples are taken at a time point prior to initiation of the treatment and one or more second samples are taken at a time point during or after the treatment.
5. The method of Embodiment 3, wherein one or more first samples are taken at a time point during the treatment and one or more second samples are taken at a time point during the treatment subsequent to the time point of the one or more first samples.
6. The method of Embodiment 3, wherein one or more first samples are taken at a time point during the treatment and one or more second samples are taken at a time point after the treatment has been discontinued.
7. A method for selecting a subject suffering from a condition associated with hypoxia, to receive therapeutic treatment to treat the condition, the method comprising determining the level of cell free RNA of at least one p53 inducible gene in a biological sample obtained from the subject and selecting the subject to receive said therapeutic treatment if the level of cell free RNA of at least one p53 inducible gene is above or below a predetermined range associated with the at least one p53 inducible gene.
8. A kit for performing a method according to any one of Embodiments 1 to 7, comprising at least one reagent for amplifying a cell free RNA of at least one p53 inducible gene from a biological sample, and instructions for performing the method of any one of claims 1 to 7.
9. The kit of Embodiment 8, wherein said at least one reagent comprises a primer or a probe for specifically hybridizing with said at least one p53 inducible gene.
10. The kit of Embodiments 8 or 9, said kit further comprising at least one reagent for extracting cell-free RNA from a biological sample
11. The method of any one of Embodiments 1 to 7 or kit of any one of Embodiments 8 to 10, wherein said sample is a bodily fluid sample.
12. The method or kit of Embodiment 11, wherein said bodily fluid sample is a blood sample.
13. The method or kit of Embodiments 11, wherein said sample is a serum sample.
14. The method or kit of Embodiment 11, wherein said sample is a plasma sample.
15. The method of any one of Embodiments 1 to 7 or kit of any one of Embodiments 8 to 10, wherein the level of cell free RNA of at least one p53 inducible gene is determined by RT-PCR.
16. The method or kit of Embodiment 15, wherein said RT-PCR is real-time quantitative RT-PCR.
17. The method of any one of Embodiments 1 to 7 or kit of any one of Embodiments 8 to 10, wherein the condition associated with hypoxia is selected from cardiovascular diseases, cancer, cerebrovascular accident (CVA) and fetal stress.
18. The method or kit of Embodiment 17, wherein the condition associated with hypoxia is fetal stress.
19. The method or kit of Embodiment 17, wherein the cardiovascular diseases is myocardial infarction.
20. The method or kit of Embodiments 18 or 19, wherein the at least one p53 inducible gene is selected from TP53 (GeneBank Accession No. Nm_{—}000546), p21 (GeneBank Accession No. Nm_{—}000389), ERCC5 (GeneBank Accession No. Nm_{—}000123), MDM2 (GeneBank Accession No. Nm_{—}0006878), TP53I3 (GeneBank Accession No. Nm_{—}004881), NOTCH1 (GeneBank Accession No. Nm_{—}017617), PIGF (GeneBank Accession No. Nm_{—}002643), BTG2 (GeneBank Accession No. Nm_{—}006763), ZMAT3 (GeneBank Accession No. Nm_{—}0022470), APAF1 (GeneBank Accession No. Nm_{—}013229), FAS (GeneBank Accession No. Nm_{—}152873), ANGPTL2 (GeneBank Accession No. Nm_{—}012098), PUMA (GeneBank Accession No. Nm_{—}014417), IGFBP6 (GeneBank Accession No. Nm_{—}002178), GDF15 (GeneBank Accession No. Nm_{—}004864), BNIP3L (GeneBank Accession No. Nm_{—}004331.2), TGFβ33 (GeneBank Accession No. Nm_{—}003239), VEGF (GeneBank Accession No. Nm_{—}001025366) and HIF-1α.
21. The method or kit of Embodiment 20, wherein the at least one p53 inducible gene is selected from p21 (GeneBank Accession No. Nm_{—}000389), BTG2 (GeneBank Accession No. Nm_{—}006763), HIF-1α (GeneBank Accession No. Nm_{—}001530), NOTCH1 (GeneBank Accession No. Nm_{—}017617), TGFβ3 (GeneBank Accession No. Nm_{—}003239) and ZMAT3 (GeneBank Accession No. Nm_{—}0022470).

22. The method or kit of Embodiment 20, wherein the at least one p53 inducible gene is p21 (GeneBank Accession No. Nm_{—}000389).
23. The method or kit of Embodiment 20, wherein the at least one p53 inducible gene is BTG2 (GeneBank Accession No. Nm_{—}006763).

### DETAILED DESCRIPTION OF SOME NON-LIMITING EMBODIMENTS

The present invention is based on the finding that a change in the concentration of cell free RNA of various p53 inducible genes, particularly in the blood, is correlated with various conditions associated with hypoxia, such as fetal stress (reflected by low oxygen levels measured in the fetus), preeclampsia, ischemic heart disease, stroke (cerebrovascular accident (CVA)) and myocardial infarction.

Thus, without wishing to be bound by theory, hypoxia is known to affect p53 and p53 inducible gene expression levels.

Based on the above, the inventors of this invention have envisaged that cell free RNA can be used as a valid diagnostic tool for predicting various diseases associated with hypoxia.

The present invention also contemplates a tool for assessing the effectiveness of treatment of a condition associated with hypoxia based on the difference in the level of cell free RNA of various p53 inducible genes before and after treatment of the condition associated with hypoxia.

Thus, in accordance with a first of its aspects, there is provided a method for detecting a condition associated with hypoxia in a subject, the method comprising detecting in a biological sample obtained from the subject the level of a cell free Ribonucleic acid (RNA) of at least one p53 inducible gene, wherein a level of the cell free RNA above or below a predetermined range associated with the at least one p53 inducible gene, is indicative that the subject has a condition associated with hypoxia.

As used herein the term “detecting” refers to quantitative as well as qualitative determination of the presence or absence of cell free RNA in a biological sample obtained from a subject. The detection thus allows determining the existence (or non-existence) of a pathological condition associated with hypoxia (e.g. myocardial ischemia; acute (first few hours to 7 days), healing (7 to 28 days) and healed (29 days and beyond) stages of myocardial infarction) in a subject, based upon the level of the cell free RNA in the biological sample obtained from a subject.

As used herein the term “condition associated with hypoxia” refers to a condition in which oxygen level is reduced below a pre-determined normal physiological level or range in an organ or tissue, generally as a result of reduced blood flow to the organ. This reduction in blood flow may result from the following non-limiting circumstances: (i) blockage of a vessel by an embolus (blood clot); (ii) blockage of a vessel due to atherosclerosis; (iii) breakage of a blood vessel (a bleeding stroke); (iv) blockage of a blood vessel due to vasoconstriction such as occurs during vasospasms and possibly, during transient ischemic attacks (TIA) and following subarachnoid hemorrhage. Hypoxia according to the present teachings may be chronic or transient.

Conditions which are associated with hypoxia may include, but are not limited to, cerebrovascular accident (CVA), fetal stress (e.g. compromise of the fetus during the antepartum period (before labor) or intrapartum period (birth process); interchangeable with fetal hypoxia (low oxygen levels in the fetus)), cardiovascular diseases and conditions such as acute coronary syndromes, ischemic heart disease, myocardial ischemia (also known as ischemic heart disease), myocardial infarction (MI) (including all stages thereof, as described herein) cardiac surgery, neurosurgery, cerebral hypoxia, cerebral infarction, surgery (e.g., per-surgical hypoxia, post-operative hypoxia), trauma, pulmonary disease, pulmonary hypertension, chronic obstructive pulmonary disease (COPD), coronary artery disease, peripheral vascular disease [e.g., arteriosclerosis (atherosclerosis, transplant accelerated arteriosclerosis), deep vein thrombosis], cancer (such as but not limited to, cervical, colon, renal, lung, uterine, breast or pancreatic cell carcinoma, lymphoma, leukemia), angiogenesis and angiogenesis related disorders (such as but not limited to, diabetic retinopathy, macular degeneration, psoriasis and rheumatoid arthritis), renal failure, skeletal muscle ischemia, sleep apnea, hypoxia during sleep, viral infection, bacterial infection, smoking, anemia, hypovolemia, hemorrhage, hypertension, diabetes, vasculopathologies, Reynaud's disease, endothelial dysfunction, regional perfusion deficits (e.g., limb, gut, renal ischemia), thrombosis, frost bite, decubitus ulcers, asphyxiation, poisoning (e.g., carbon monoxide, heavy metal), altitude sickness, sudden infant death syndrome (SIDS), asthma, congenital circulatory abnormalities (e.g., Tetralogy of Fallot) and Erythroblastosis (blue baby syndrome).

As used herein the term “biological sample” refers to any sample obtained from a subject. Preferable, such a sample is a bodily fluid. Samples which qualify include, but are not limited to, blood, plasma, serum, amniotic fluid, sputum, saliva, semen, urine, feces, bone marrow and cerebrospinal fluid (CSF). The term “serum” refers to the fluid portion of the blood obtained after removal of the fibrin clot and blood cells, distinguished from the plasma in circulating blood. The term “plasma” refers to the fluid, non-cellular portion of the blood, distinguished from the serum obtained after coagulation. In some embodiments biological sample is selected from sputum or saliva. The samples are typically treated to remove therefrom cellular fractions, i.e. to become a cell free RNA sample, as further discussed below.

Procedures for obtaining biological samples from subjects are well known in the art. Such procedures include, but are not limited to, blood sampling, amniocentesis, chorionic villus sampling and urine collection.

As used herein, “subject” refers to any warm-blooded animal, particularly including a member of the class mammalian such as, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex and, thus, includes adult and newborn subjects, whether male or female. The subject in the context of the invention is preferably a human subject.

As used herein the phrase “cell free ribonucleic acid (RNA)” (also called circulating RNA or ciRNA) refers to such RNA (e.g. mRNA) present within the cell-free fraction of a sample. The cell-free RNA described herein is not comprised in intact cells (i.e., comprising uncompromised plasma membrane) but is typically associated with particles (e.g. placenta-derived syncytiotrophoblast microparticles, see Rusterholz et al., supra; or apoptotic bodies, see Hasselmann et al., Clin Chem (2001) 47:1488-1489). In some embodiments the cell-free RNA is intact (i.e. not fragmented).

Cell-free RNA samples may be extracted from the biological sample according to any method known in the art (see general materials and methods section of the Examples section). For instance, after obtaining the biological sample (i.e. blood) the sample is prepared as was previously described (see for Example Ng et al., supra). Briefly, all nucleated cells are removed from the sample by two centrifugation cycles (e.g. at 1,600×g for 10 minutes at 4° C.). The resultant cell-free sample (e.g. plasma or serum) is transferred to a clean tube (e.g. eppendorf tube), mixed with TRIzol LS reagent (Invitrogen, Carlsbad, Calif.) plus chloroform and centrifuged (e.g. at 11,900×g for 15 minutes at 4° C.). The aqueous layer is transferred into a new tube and mixed with 70% ethanol (at a 1:1 ratio). The mixture is then transferred to an RNeasy minicolumn (RNeasy mini kit, Qiagen, Valencia, Calif.) according to the manufacturer's recommendations and total RNA is eluted in RNase-free water.

The quantification of cell free RNA, according to the present invention can be achieved by any methods or kits employing such methods known in the art. Some none limiting examples of such methods include reverse transcription-polymerase chain reaction (RT-PCR), real-time RT-PCR (such as TaqMan® and Assays-on-Demand™, (Applied Biosystems, Foster City, Calif., USA), Molecular Beacons, Scorpions® and SYBR® Green (Molecular Probes)), plasma/serum circulating RNA purification kits (such as Norgen's; www.norgenbiotek.com) and RNA microarray. In some embodiments the cell free RNA is further normalized against several housekeeping genes (e.g. beta-actin, GAPDH, CypA, HPRT, Ki-67, SDHA, HPRT1, HBS1L, AHSP, beta-2-microglobulin) in order to provide a more accurate measurement of the cell free RNA in the biological sample. Reagents for carrying out RNA quantification typically comprise at least one primer and/or probe for specifically hybridizing with the at least one p53 inducible gene. As used herein, the term “specifically hybridizing” refers to forming a double strand molecule such as RNA:RNA, RNA:DNA and/or DNA:DNA molecules.

Amplifying and/or detecting the cell-free RNA of a specific gene typically involve the use of at least one sequence specific oligonucleotide (see general materials and methods section of the Examples section which follows). The oligonucleotides may be of at least 10, at least 15, at least 20, at least 25, or at least 30 bases specifically hybridizable with polynucleotide sequences of the present invention.

Detection of hybrid duplexes can be carried out using a number of methods, including by sequence specific probes (e.g. MGB-probes). Typically, a label or tag is attached (conjugated) to the probe. Such labels or tags are of standard use in the art and include radioactive, fluorescent, biological or enzymatic tags or labels.

Traditional hybridization assays include PCR, RT-PCR, RNase protection; in-situ hybridization, primer extension, Northern Blot and dot blot analysis (see Examples section hereinbelow).

Examples of specific primers and probes suitable for the detection of certain p53 inducible genes are provided in the Examples section below. Any person skilled in the art would be able to generate suitable primers and probes based on the available gene sequences provided herein or available in the art using methods well known in the art.

As used herein the term “p53 inducible gene” refers to a gene wherein the expression of the gene is regulated either directly or indirectly by protein 53 (p53). In some embodiments regulation of the gene by p53 occurs by mean of transcriptional regulation whereby the change in the gene's expression level depends on transcription rates of the gene (e.g. by affecting transcription initiation). When the gene is regulated at the transcription level the p53 protein may regulate the expression of the gene by binding to a DNA binding site which is sometimes located near the promoter of the gene or by binding of a regulatory binding site to switch the gene on (i.e. activate the gene) or to shut off a gene (i.e. repress the gene). In some embodiments, regulation of the gene occurs at the level of one or more of post-transcriptional modification (e.g. glycosylation, acetylation, fatty acylation, disulfide bond formations, etc.), RNA transport, RNA translation (e.g. translation initiation), protein transport, protein stability, mRNA degradation (i.e. transcript Stability), affecting a chromatin component of the gene (e.g. affect accessibility of the chromatin to RNA polymerases and transcription factors).

As used herein the term “predetermined range associated with the at least one p53 inducible gene” generally refers to a concentration range of p53 inducible genes which defines the level of cell free RNA measured in samples obtained from healthy subjects not suffering from any condition associated with hypoxia, i.e. normal, control, hypoxia-unaffected samples. Such a control sample is typically obtained from a subject of the same age range, physiological state (e.g., pregnancy) and gender. Under certain circumstances it may even be derived of the same subject prior to the state of hypoxia, e.g. in subjects that are susceptible to developing a condition associated with hypoxia (e.g. before pregnancy). In some embodiments the predetermined range is a concentration range of p53 inducible genes which defines the level of cell free RNA measured from samples obtained from subjects in various stages of a condition associated with hypoxia (e.g. for MI—acute, healing or healed stages of myocardial infarction, as described herein). The predetermined range may be determined experimentally (e.g. by sampling cell free RNA from blood obtained from MI patients) or derived from the literature if available.

Thus, according to the present invention, the level of the cell free RNA measured to be statistically different (i.e. above or below) from a predetermined concentration range, as defined above, of at least one p53 inducible gene, is indicative that the subject has a condition associated with hypoxia.

In some embodiments the condition associated with hypoxia is selected from fetal stress, arteriosclerotic vascular disease, myocardial ischemia, myocardial infarction, unstable angina, sudden cardiac death, coronary plaque rupture, or thrombosis in all stages of their occurrence.

As used herein, the term “ischemia” refers to a condition which involves insufficient supply of blood to an organ, usually due to a blocked artery. As used herein, the term “myocardial ischemia” refers to a disorder of cardiac function caused by insufficient blood flow to the muscle tissue of the heart. Ischemia can be silent or symptomatic. The decreased blood flow may, for example, be due to narrowing of the coronary arteries (coronary arteriosclerosis), to obstruction by a thrombus (coronary thrombosis), or less commonly, to diffuse narrowing of arterioles and other small vessels within the heart. As used herein, the term “myocardial infarction (MI)” (also known as acute myocardial infarction (AMI) or heart attack), refers to the irreversible necrosis of heart muscle secondary to prolonged ischemia. Typically, a myocardial infarction is caused by an occlusion or blockage of arteries supplying the muscles of the heart and results in injury or necrosis of the heart muscle (i.e. heart attack). In the context of the present invention myocardial infarction refers to any stage of the disease (e.g. acute, healing or healed stages of myocardial infarction, as described herein). As used herein, the term “fetal stress” refers to any condition in which the fetus is at risk of developing a pregnancy related complication. Fetal stress includes, without being limited to, inadequate nutrient supply and cessation of fetal growth. Fetal stress may affect fetal development and brain functions and plays a significant role in pregnancy outcomes related to prematurity and urgent deliveries (e.g. c-section). Conditions associated with fetal stress include, but are not limited to, abnormal pregnancy, fetal hypoxia, fetal stress, intrauterine growth retardation (IUGR), fetal growth restriction (FGR), fetal alcohol syndrome (FAS), nicotine intake, alcohol intake, inadequate nutrition, maternal diabetes, advanced maternal age and excessive maternal exercise. Additional examples of pregnancy associated hypoxic conditions associated with fetal stress are exemplified in detail hereinabove.

In some embodiments the fetal stress, as defined herein, is associated with hypoxia being related to a pregnancy associated hypoxic condition such as preeclampsia, eclampsia, mild preeclampsia, chronic hypertension, EPH gestosis, gestational hypertension, superimposed preeclampsia (including preeclampsia superimposed on chronic hypertension, chronic nephropathy or lupus), HELLP syndrome (hemolysis, elevated liver enzymes, low platelet count), nephropathy, gestational diabetes, placental hypoxia, fetal hypoxia, intrauterine growth retardation (IUGR), fetal growth restriction (FGR), fetal alcohol syndrome (FAS).

In some embodiments, the at least one p53 inducible gene is selected from p21 (GeneBank Accession No. U09579), VEGF (GeneBank Accession No. NM_{—}001025366), HIF1α (GeneBank Accession No. NM_{—}001530.2), MDM2 (EST=MDM2, GeneBank Accession No. M92424) and TP53I3 (GeneBank Accession No. NM_{—}147184.1), Fas antigen/TNFR6 (GeneBank Accession No. X89101), TNFR18 (GeneBank Accession No. AI923712), Gelsolin (GeneBank Accession No. X04412), btg1 (GeneBank Accession No. X61123), EST=PIG8 (Etoposide induced, GeneBank Accession No. R11732), T10 mRNA/human sentrin/SUMO (GeneBank Accession No. U83117), Apaf1 (GeneBank Accession No. AL135220), ANGL2, EST=Angiopoietin like (GeneBank Accession No. AF125175), and cytosolic adenylate kinase (GeneBank Accession No. J04809), S100 calcium-binding protein A4 (GeneBank Accession No. M80563), cyclin G2 (GeneBank Accession No. U47414), EST=Ras inhibitor Rin1 (GeneBank Accession No. L36463), B-cell translocation gene 2, anti-proliferative (GeneBank Accession No. U72649), ERCC5 (GeneBank Accession No. AW502004), H2B and H2A histone genes (291A, GeneBank Accession No. Z83336), Notch gene homolog 1, (Drosophila GeneBank Accession No. AI566271), Eph receptor A2 (GeneBank Accession No. M59371), hepatocyte growth factor-like protein gene (GeneBank Accession No. U37055) and EST=Alpha-L Fucosidase precursor (GeneBank Accession No. M29877), Mannosidase 2, alpha B1 (GeneBank Accession No. U37248), phosphomannomutase Sec53p homolog (GeneBank Accession No. U86070), Spermidine (GeneBank Accession No. U40369), peroxisomal integrel membrane protein PMP34 (GeneBank Accession No. AI871429), hypothetical protein/human Cubilin (GeneBank Accession No. AF034611), Arginosuccinate synthetase 1 (GeneBank Accession No. AA069289), Lipocortin 1/human Annexin A1 (GeneBank Accession No. ASW379702), EST=Xanthine dehydrogenase (GeneBank Accession No. U39487) and EST=Prostaglandin synthase (GeneBank Accession No. M98539), Biotin Carboxylase/human neuronal acidic protein (GeneBank Accession No. AI422580), Ly-6 alloantigen/human dihydropyrimidinase (GeneBank Accession No. D78014), neural visinin-like protein 3 (NVP-3, GeneBank Accession No. AI391924), EST=Aryl-hydrocarbon receptor interacting protein (GeneBank Accession No. U78521), PIGF, Phosphatidylinositol glycan, class F 3A (GeneBank Accession No. WO15279), EST=UNC-51 like kinase 1 (GeneBank Accession No. AL046256), DNA for tob family/transducer of ERBB2 (GeneBank Accession No. D38305), TYRO protein tyrosine kinase binding protein (GeneBank Accession No. AI299346), p53-inducible zinc finger protein (Wig-1/ZMAT3) mRNA (GeneBank Accession No. AI457344) and friend of GATA-1 (FOG) 1 (GeneBank Accession No. AF488691), T-complex-associated testis expressed 3 (GeneBank Accession No. AA781436), Selenium binding protein 1 (GeneBank Accession No. U29091), EST=BPM1/human Plectin 1 (GeneBank Accession No. Z54367), EST=Vamp2/human synaptobrevin 2 (GeneBank Accession No. M36205), Fas/APO-1 cell surface antigen (GeneBank Accession No. X63717), Bcl-2 binding component 3 (bbc3/PUMA, GeneBank Accession No. U82987), Bcl-6 (GeneBank Accession No. U00115), Bak (GeneBank Accession No. U16811), ATL derived PMA responsive peptide (GeneBank Accession No. D90070) and GADD45 (GeneBank Accession No. M60974), BTG2 (GeneBank Accession No. U72649), Damage-specific DNA binding protein (GeneBank Accession No. U18300), Histone 2A-like protein (GeneBank Accession No. U90551), PCNA (GeneBank Accession No. M15796), Endoglin (GeneBank Accession No. X72012), Versican (GeneBank Accession No. U16306), Heavy chain 4F2 (GeneBank Accession No. M21904), SMAD7 (GeneBank Accession No. AF010193), TGF-Beta Superfamily protein (GeneBank Accession No. AB000584) and IGFBP6 (GeneBank Accession No. M62402), Quiescin/QSCN6 (GeneBank Accession No. L42379), Adipophilin (GeneBank Accession No. X97324), Multiple exostoses type II protein (GeneBank Accession No. U72263), Vascular smooth muscle alpha-actin (GeneBank Accession No. X13839), Smoothelin (GeneBank Accession No. Z49989), Neurofilament subunit NF-L (GeneBank Accession No. X05608), NB Thymosin beta (GeneBank Accession No. D82345), LIM domain protein (GeneBank Accession No. X93510), Lysyl oxidase-like protein (GeneBank Accession No. U24389) and UDP-Galactose 4 Epimerase (GALE, GeneBank Accession No. L38668), cAMP activated Protein Kinase B (GeneBank Accession No. Y12556), Lysosomal Mannosidase alpha B (GeneBank Accession No. U05572), Carboxylesterase (liver, GeneBank Accession No. Y09616), ABC3 (GeneBank Accession No. U78735), Apolipoprotein C-I (VLDL, GeneBank Accession No. M20902), CART (GeneBank Accession No. U20325), Lecithin-cholesterol acyltransferase (GeneBank Accession No. M12625), Rhodanese (GeneBank Accession No. D87292), NECDIN related protein (GeneBank Accession No. U35139) and NSCL-2 gene (GeneBank Accession No. M96740), FEZI-T gene (GeneBank Accession No. U60062), Ninjurin 1 (GeneBank Accession No. U72661), Amyloid precursor-like protein (GeneBank Accession No. U48437), c-Ha-ras (GeneBank Accession No. J00277), Intestinal VIPR related protein (GeneBank Accession No. X77777), Diacylglycerol Kinase (alpha, GeneBank Accession No. X62535), Putative ser/thr protein kinase (GeneBank Accession No. U56998), DM Kinase (GeneBank Accession No. L08835), DRAL-FHL2 (GeneBank Accession No. L42176) and Activating transcription factor 3 (GeneBank Accession No. L19871), ZNF127-Xp (GeneBank Accession No. U38315), LISCH7 (GeneBank Accession No. AD000684), Zinc finger protein 7 (GeneBank Accession No. M29580), Tip-1 (GeneBank Accession No. U90913), Nuclear factor NF-116 (GeneBank Accession No. HG3494), POM-ZP3 (GeneBank Accession No. U10099), KIAAA0247 (GeneBank Accession No. D87434), Mammaglobin 1 (GeneBank Accession No. U33147), Disulfide isomerase related protein (GeneBank Accession No. J05016) and OS4 (GeneBank Accession No. U81556), Infertility-related sperm protein (GeneBank Accession No. S58544), KIAA0147 (GeneBank Accession No. D63481), SURF-1 (GeneBank Accession No. Z35093), WD repeat protein HAN11 (GeneBank Accession No. U94747), p53-induced gene 3 (PIG3, GeneBank Accession No. AF010309), MIC-1/GDF15, member of TGF-L family (GeneBank Accession No. AF019770), DDB2, involved in nucleotide excision repair (GeneBank Accession No. U18300a), MYD88, myeloid differentiation (GeneBank Accession No. U70451a), Retinoic acid receptor L (GeneBank Accession No. X07282) and Fas/APO1 (GeneBank Accession No. Z70519), MAPK14 (GeneBank Accession No. L35253), Bcl2-associated X protein (Bax, GeneBank Accession No. L22474), FKBP4 (possible peptidyl-prolyl-cis-trans-isomerase, GeneBank Accession No. M88279), Mitochondrial stress 70 precursor (Mortalin2, GeneBank Accession No. L15189a), p57KIP2 (CDK inhibitor 1C, GeneBank Accession No. U22398), DNA ligase 1 (LIG1, GeneBank Accession No. M36067), DNA excision repair-related 1 (ERCC5, GeneBank Accession No. L20046a), G/T mismatch thymine DNA glycosylase (TDG, GeneBank Accession No. U51166), Homeobox protein 1 (HOXD3, GeneBank Accession No. D111117a) and MAP4K5 (activator of Jun N-terminal kinase, GeneBank Accession No. U77129, Replication factor A protein 1 (RPA1, GeneBank Accession No. M63488), Bc12 antagonist/killer 1 (BAK1, GeneBank Accession No. U23765), TGF-L inducible early growth response gene (TIEG, GeneBank Accession No. S81439a), MAP2K1 (MEK1 1.5 Kinase, GeneBank Accession No. L05624), Chondroitin sulfate proteoglycan 2 (CSPG2, GeneBank Accession No. U16306a) and Zinc finger protein 197 (p18 protein, ZNF197, GeneBank Accession No. Z21707a), Adenylate Kinase 3 (GeneBank Accession No. J04809), Aldolase A (GeneBank Accession No. NM_{—}000034), Aldolase C (GeneBank Accession No. NM_{—}0051654), Enolase 1 (ENOL, GeneBank Accession No. NM_{—}001428), Glucose Transporter 1 (GeneBank Accession No. NM_{—}153369), Glucose Transporter 3 (GeneBank Accession No. NM_{—}001009770), Glyceraldehyde-3-phosphate Dehydrogenase (GeneBank Accession No. NM_{—}002046), Hexokinase 1 (GeneBank Accession No. Nm_{—}000188×) and Hexokinase 2 (GeneBank Accession No. Nm_{—}000189X), Insulin-like Growth Factor 2 (IGF-2, GeneBank Accession No. NM_{—}000612), IGF Binding Protein 1 (IGFBP-1, GeneBank Accession No. uc001gkz.1), IGFBP-3 (GeneBank Accession No. uc003tnr.1), Lactate Dehydrogenase A (GeneBank Accession No. NM_{—}005566), Phosphoglycerate Kinase 1 (GeneBank Accession No. NM_{—}000291), Pyruvate Kinase M (GeneBank Accession No. M23725), Transforming Growth Factor β3 (TGF β3, GeneBank Accession No. uc001doh.1), Ceruloplasmin (GeneBank Accession No. NM_{—}000096), Erythropoietin (GeneBank Accession No. NM_{—}000799), Transferrin (GeneBank Accession No. NM_{—}001063) and Transferrin Receptor (GeneBank Accession No. NM_{—}003234), α1B-Adrenergic Receptor (GeneBank Accession No. NM_{—}000679), Adrenomedullin (GeneBank Accession No. NM_{—}001124), Endothelin-1 (GeneBank Accession No. NM_{—}001101696), Heme Oxygenase 1 (GeneBank Accession No. NM_{—}002133), Nitric Oxide Synthase 2 (GeneBank Accession No. uc002gzu.1) and VEGF Receptor FLT-1 (GeneBank Accession No. NM_{—}001025366) and BCL2/adenovirus E1B 19 kd-interacting protein 3-like (BNIP3L, GeneBank Accession No. NM_{—}004331.2.

In some embodiments the p53 inducible genes are selected from TP53, P21, ERCC5, MDM2, TP53I3 (PIG3), NOTCH, PIGF, BTG2, ZMAT3 (WIG1), APAF1, FAS, ANGPTL2, PUMA (BBC3), IGFBP6, GDF15, BNIP3L, TGF-133, VEGF, HIF1α as depicted in Table 1 and are quantified using the TaqMan® Gene Expression Assays (Applied Biosystems) also as shown in Table 1, or using equivalent commercial or designed primer/probe sets, as recognized by the skilled artisan.

In some embodiments the p53 inducible genes are selected from TP53, P21, ERCC5, MDM2, TP53I3 (PIG3), NOTCH, PIGF, BTG2, ZMAT3 (WIG1), APAF1, FAS, ANGPTL2, PUMA (BBC3), IGFBP6, GDF15, BNIP3L, TGF-β3, VEGF, HIF1α as depicted in Table 3 and are quantified using the Assays-on-Demand™, Applied Biosystems also as shown in Table 3, or using equivalent commercial or designed primer/probe sets, as recognized by the skilled artesian.

TABLE 1
p53 inducible genes

| | Gene symbol | Accession # | SEQ ID NO | Assay # |
|---|---|---|---|---|
| 1 | TP53 | Nm_000546 | SEQ ID NO: 16 | Hs01034249_m1 |
| 2 | P21 (CDKN1A) | Nm_000389 | SEQ ID NO: 17 | Hs01121168_m1 |
| 3 | ERCC5 | Nm_000123.2 | SEQ ID NO: 18 | Hs01557031_m1 |
| 4 | MDM2 | Nm_006878 | SEQ ID NO: 19 | Hs00234753_m1 |
| 5 | TP53I3 (PIG3) | Nm_004881 | SEQ ID NO: 20 | Hs00153280_m1 |
| 6 | NOTCH1 | Nm_017617 | SEQ ID NO: 21 | Hs00413187_m1 |
| 7 | PIGF | Nm_002643 | SEQ ID NO: 22 | Hs00601696_m1 |
| 8 | BTG2 | Nm_006763 | SEQ ID NO: 23 | Hs00198887_m1 |
| 9 | ZMAT3 (WIG1) | Nm_022470 | SEQ ID NO: 24 | Hs01074692_m1 |
| 10 | APAF1 | Nm_013229 | SEQ ID NO: 25 | Hs00185508_m1 |
| 11 | FAS | Nm_152873 | SEQ ID NO: 26 | Hs00910107_m1 |
| 12 | ANGPTL2 | Nm_012098 | SEQ ID NO: 27 | Hs00765775_m1 |
| 13 | PUMA (BBC3) | Nm_014417 | SEQ ID NO: 28 | Hs00248075_m1 |
| 14 | IGFBP6 | Nm_002178 | SEQ ID NO: 29 | Hs00181853_m1 |
| 15 | GDF15 | Nm_004864 | SEQ ID NO: 30 | Hs00171132_m1 |
| 16 | BNIP3L | NM_004331.2 | SEQ ID NO: 31 | Hs00188949_m1 |
| 16 | TGFβ3 | Nm003239 | SEQ ID NO: 32 | Hs00234245_m1 |
| 17 | VEGF | Nm_001025366 | SEQ ID NO: 33 | Hs99999070_m1 |
| 18 | HIF1a | Nm_001530 | SEQ ID NO: 34 | Hs00936372 |

TABLE 2
Primers and probes

| Gene | Forward | Reverse | MGB- |
|---|---|---|---|
| name | primer | primer | probe |
| p21 | CTGGAGACTCT | CGGCGTTTGG | CAGACCAG |
| GeneBank | CAGGGTCGAA | AGTGGTAGA | CATGACAG |
| Accession | (SEQ ID | (SEQ ID | (SEQ ID |
| No. U09579 | NO: 1) | NO: 2) | NO: 3) |
| MDM2 | GACTCCAAGC | ACATGTTGGTAT | CGGATGGT |
| GeneBank | GCGAAAACC | TGCACATTTGC | GAGGAGC |
| Accession | (SEQ ID | (SEQ ID | (SEQ ID |
| No. M92424 | NO: 4) | NO: 5) | NO: 6) |
| VEGF | CTACCTCCACC | TGCGCTGATAG | AGGCTGCA |
| GeneBank | ATGCCAAGTG | ACATCCATGA | CCCATG |
| Accession No. | (SEQ ID | (SEQ ID | (SEQ ID |
| NM_001025366 | NO: 7) | NO: 8) | NO: 9) |
| HIF1α | GCATCTTG | CCATCCAA | AGCTATT |
| GeneBank | ATAAGGCC | GGCTTTCA | TGCGTGT |
| Accession | TCTGTGA | AATAAAA | GAGGA |
| No. | (SEQ ID | (SEQ ID | (SEQ ID |
| NM_001530.2 | NO: 10) | NO: 11) | NO: 12) |
| TP53I3 | GCAACGCTGAA | TAGGATCCGCCT | TGCTGGAGT |
| GeneBank | ATTCACCAAA | ATGCAGTCTAG | TAATCTTAT |
| Accession No. | (SEQ ID | (SEQ ID | (SEQ ID |
| NM_147184.1 | NO: 13) | NO: 14) | NO: 15) |

TABLE 3
Assays-on-Demand ™, Applied Biosystems

| | | | | | | Assay |
|---|---|---|---|---|---|---|
| | | | | | Fold | location |
| | | | | | increase by | (Exon-Exon |
| | Gene symbol | Accession # | SEQ ID NO: | Assay # | p53 | boundaries) |
| 1 | TP53 | Nm_000546 | SEQ ID NO: 16 | Hs01034249_m1 | — | |
| 2 | p21 (CDKN1A) | Nm_0003891 | SEQ ID NO: 17 | Hs01121168_m1 | 21 | 1-2 |
| 3 | ERCC5 | Nm_000123.2 | SEQ ID NO: 18 | Hs01557031_m1 | 58 | 1-2 |
| 4 | MDM2 | Nm_006878 | SEQ ID NO: 19 | Hs00234753_m1 | 14 | 1-2 |
| 5 | TP53I3 (PIG3) | Nm_004881 | SEQ ID NO: 20 | Hs00153280_m1 | 11 | 4-5 |
| 6 | NOTCH1 | Nm_017617 | SEQ ID NO: 21 | Hs00413187_m1 | 26 | 4-5 |
| 7 | PIGF | Nm_002643 | SEQ ID NO: 22 | Hs00601696_m1 | 15 | 1-2 |
| 8 | BTG2 | Nm_006763 | SEQ ID NO: 23 | Hs00198887_m1 | 10 | 1-2 |
| 9 | ZMAT3 | Nm_022470 | SEQ ID NO: 24 | Hs01074692_m1 | 24 | 2-3 |
| | (WIG1) | | | | | |
| 10 | APAF1 | Nm_013229 | SEQ ID NO: 25 | Hs00185508_m1 | 7 | 18-19 |
| 11 | FAS | Nm_152873 | SEQ ID NO: 26 | Hs00910107_m1 | 54 | 3-4 |
| 12 | ANGPTL2 | Nm_012098 | SEQ ID NO: 27 | Hs00765775_m1 | 37 | 3-4 |
| 13 | PUMA(BBC3) | Nm_014417 | SEQ ID NO: 28 | Hs00248075_m1 | 30 | 3-4 |
| 14 | IGFBP6 | Nm_002178 | SEQ ID NO: 29 | Hs00181853_m1 | 30 | 1-2 |
| 15 | GDF15 | Nm_004864 | SEQ ID NO: 30 | Hs00171132_m1 | 32 | 1-2 |
| 16 | BNIP3L | Nm_004331.2 | SEQ ID NO: 31 | Hs00188949_m1 | 16 | 2-3 |
| 17 | TGFB3 | Nm_003239 | SEQ ID NO: 32 | Hs00234245_m1 | p53- | 1-2 |
| | | | | | associated | |
| 18 | VEGF | Nm_001025366 | SEQ ID NO: 33 | Hs99999070_m1 | p53- | 3-3 |
| | | | | | assocoated | |
| 19 | HIF1a | Nm_001530 | SEQ ID NO: 34 | Hs00936372 | p53- | 2-3 |
| | | | | | associated | |

In accordance with a further aspect, the present invention provides a method for determining the severity of a condition associated with hypoxia in a subject comprising determining the level of a cell free RNA of at least one p53 inducible gene in a biological sample obtained from the subject and comparing the level of the cell free RNA of the p53 inducible gene with a predetermined value (which may be a discrete number of a range) that correlates with the level of the at least one p53 inducible gene with the severity of the a condition associated with hypoxia, the comparison allowing determination of the severity of the condition associated with hypoxia in the subject.

In accordance with a further aspect, there is provided by the present invention a method for determining the effectiveness of a therapeutic treatment of a condition associated with hypoxia in a subject comprising determining the level of cell free RNA of at least one p53 inducible gene from two or more biological samples obtained from the subject, at two or more successive time points, at least one of the time points is during or after the treatment, wherein:
(i) for a p53 inducible gene that is over-expressed in a condition associates with hypoxia, a decrease in the level of the cell free RNA of the p53 inducible gene between the two or more samples, is indicative of effectiveness of the therapeutic treatment;
(ii) for a p53 inducible gene that is repressed in a condition associates with hypoxia, an increase in the level of the cell free RNA of the p53 inducible gene between the two or more samples, is indicative of effectiveness of the therapeutic treatment.

As used herein the term “effectiveness of a therapeutic treatment” refers to the assessment of the success of treating a subject having a condition associate with hypoxia (e.g. myocardial infarction) by measuring the improvement in the health condition of the subject being treated for a condition associate with hypoxia. In accordance with the present invention, the assessment of the subject's medical health can be carried out using any acceptable medical test/procedure known in the art.

In some embodiments, the effectiveness of a therapeutic treatment is manifested by the return of at least one p53 inducible gene expression level to a normal gene expression level, namely the level of expression of said at least one p53 inducible gene measured in a control (e.g. a healthy subject being measured or a measurement previously obtained from a healthy subject).

As used herein a “decrease” or “increase” in the level of the cell free RNA of the p53 inducible gene refers to a statistically significant decrease or increase as measured in accordance with the invention. The determination of a statistically significant decrease or increase may be conducted using any commonly used statistical test. Those skilled in the art would know how to select the most appropriate statistical test for conducting the determination of a statistically significant decrease or increase in the level of the cell free RNA of the p53 inducible gene. In one embodiment, the test is the Chi-square test. In another embodiment the test is a t-test. In still another embodiment the test is a Mann-Whitney test.

Thus, for example, a first serum or plasma sample is obtained from a subject suffering from acute chest pain, upon admission to the hospital (e.g. between arrival at the hospital and before the beginning of a catheterization procedure or any other procedure or treatment used to treat myocardial infarction such as anti-platelet medicines, nitroglycerin, angiotensin converting enzyme inhibitors, beta-blocking agents) and additional samples are taken sequentially every few hours or days to monitor the effectiveness of the treatment (e.g. catheterization procedure or any other treatment used to cure myocardial infarction as described herein) given to the hospitalized subject.

In some embodiments, the additional samples are taken at daily (and/or hourly) intervals in the time period of between 1 to 30 days after the beginning of the treatment (e.g. catheterization procedure).

In some embodiments, the additional samples are taken between 3 to 6 hours after the beginning of the treatment (e.g. catheterization procedure).

In one embodiment, the additional samples are taken about 4 hours after the beginning of the treatment (e.g. catheterization procedure).

In some embodiments, the effectiveness of treatment is further determined by comparing the level of cell free RNA of at least one p53 inducible gene from two or more biological samples obtained from the subject, as described herein, to the RNA level of at least one p53 inducible gene obtained from a control (e.g. healthy subject) In accordance with such embodiments the effectiveness of a therapeutic treatment is assessed by comparing gene expression values of at least one p53 inducible gene, as defined herein, in subjects undergoing treatment of having completed treatment (e.g. MI patient 3 to 6 hours after a catheterization procedure), with gene expression values of at least one p53 inducible gene in a control sample (e.g. healthy subjects).

Cell free RNA is measured in these samples and the level of at least one p53 inducible gene is compared between the samples to determine the effectiveness of the treatment of the subject. In some embodiments the concentration of the cell free RNA of the at least one p53 inducible gene is also compared to a reference control sample taken from healthy individuals of similar gender, weight and age. In accordance with the present invention a reference control may also be obtained from a cell line (e.g. A2780 human ovarian cancer cell line).

In some embodiments, one or more first samples are taken at a time point prior to initiation of treatment and one or more second samples are taken at a time point during or after the treatment. In one embodiment the second sample is taken between 3 to 6 hours after treatment. In one embodiment the treatment is catheterization.

In some embodiments the one or more first samples are taken at a time point during the treatment and one or more second samples are taken at a time point during the treatment subsequent to the time point of the one or more first samples.

In some embodiments the one or more first samples are taken at a time point during the treatment and one or more second samples are taken at a time point after the treatment has been discontinued.

The one or more first samples are then compared with the one or more second samples to determine the difference between expressions of the p53 inducible gene, the comparison allowing determination of treatment effectiveness, as described herein.

In accordance with a third of its aspects, there is provided a method for selecting a subject suffering from a certain condition associated with hypoxia, to receive therapeutic treatment to treat the condition, the method comprising determining the level of cell free RNA of at least one p53 inducible gene in a biological sample obtained from the subject and selecting the subject to receive said therapeutic treatment if the level is above or below a predetermined range associated with the at least one p53 inducible gene.

In accordance with a fourth of its aspects, there is provided a kit for performing any of the methods defined herein, the kit comprising at least one reagent for amplifying a cell free RNA of at least one p53 inducible gene from a biological sample, and instructions for performing the method of the invention. In certain embodiments, the kit further comprises at least one reagent for extracting cell-free RNA from a biological sample.

In some embodiments, the at least one reagent for amplifying the cell free RNA comprises a primer or a probe for specifically hybridizing with said at least one p53 inducible gene.

In some embodiments the method of the invention comprises the steps of:
a) extracting cell free RNA from a biological sample obtained from the subject,
b) quantifying the level of the cell free RNA of at least one p53 inducible gene,
c) comparing the level of cell free RNA obtained in step b to a predetermined concentration range of the at least one p53 inducible gene and/or to a reference control.

In some embodiments, the condition associated with hypoxia is fetal stress.

In some embodiments, the condition associated with hypoxia is myocardial infarction.

In some embodiments the at least one p53 inducible gene is selected from TP53 (GeneBank Accession No. Nm_{—}000546), p21 (GeneBank Accession No. Nm_{—}000389), ERCC5 (GeneBank Accession No. Nm_{—}000123), MDM2 (GeneBank Accession No. Nm_{—}0006878), TP53I3 (GeneBank Accession No. Nm_{—}004881), NOTCH1 (GeneBank Accession No. Nm_{—}017617), PIGF (GeneBank Accession No. Nm_{—}002643), BTG2 (GeneBank Accession No. Nm_{—}006763), ZMAT3 (GeneBank Accession No. Nm_{—}0022470), APAF1 (GeneBank Accession No. Nm_{—}013229), FAS (GeneBank Accession No. Nml 52873), ANGPTL2 (GeneBank Accession No. Nm_{—}012098), PUMA (GeneBank Accession No. Nm_{—}014417), IGFBP6 (GeneBank Accession No. Nm_{—}002178), GDF15 (GeneBank Accession No. Nm_{—}004864), BNIP3L (GeneBank Accession No. Nm_{—}004331.2), TGFβ3 (GeneBank Accession No. Nm_{—}003239), VEGF (GeneBank Accession No. Nm_{—}001025366) and HIF-1α. In some embodiments the at least one p53 inducible gene is selected from p21 (GeneBank Accession No. Nm_{—}000389), BTG2 (GeneBank Accession No. Nm_{—}006763), HIF-1α (GeneBank Accession No. Nm_{—}001530), NOTCH1 (GeneBank Accession No. Nm_{—}017617), TGFβ3 (GeneBank Accession No. Nm_{—}003239) and ZMAT3 (GeneBank Accession No. Nm_{—}0022470).

In one embodiment, the primers or probes used to detect said cell free RNA of the p53 inducible genes are the primers or probes depicted in Table 2 or are primers and probes selected by the skilled artisan from primer and probes for said genes which are known from the art.

In some embodiments, the primers or probes used to detect said cell free RNA of the p53 inducible genes are the primers or probes depicted in Table 3 or are primers and probes selected by the skilled artesian from primers and probes for said genes which are known from the art.

In some embodiments the at least one p53 inducible gene is selected from p21, BTG2, TGFβ3, NOTCH1, HIF1α, MDM2 and ZMAT3.

In one embodiment the at least one p53 inducible gene is p21.

In another embodiment the at least one p53 inducible gene is BTG2.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Various embodiments and aspects of the present invention as delineated hereinabove and as claimed in the claims section below find experimental support in the following examples.

### DESCRIPTION OF SOME NON-LIMITING EXAMPLES

Reference is now made to the following examples, which together with the above description; illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Methods

Blood Collection from Pregnant Women:

15 ml blood samples were collected from healthy women with singleton (i.e. pregnancy with a single fetus) uncomplicated pregnancies and from women with complicated pregnancies. The study was approved by the Research Ethics Committee of the Sheba Medical Center. All blood samples were acquired in the Department of Obstetrics and Gynecology at the Sheba Medical Center following informed consent of the subjects.

Blood Preparation:

Blood samples were prepared as was previously described by Ng [Ng et al., Proc Natl Acad Sci (2003) 100(8):4360-2]. In detail, the blood samples were collected in EDTA-containing tubes centrifuged at 1,600×g for 10 minutes at 4° C. (to remove nucleated cells from the blood sample). Plasma and serum were then carefully transferred into 1.5 ml eppendorf tubes. The plasma samples were re-centrifuged at 16,000×g for 10 minutes at 4° C. and the supernatants were collected into fresh polypropylene tubes. The serum samples were stored at −20° C. for future reference.

RNA Extraction:

1.6 ml plasma (subsequent to centrifugation) was mixed with 2 ml TRIzol LS reagent (Invitrogen, Carlsbad, Calif.) and 0.4 ml chloroform as was previously described by Ng [Ng et al., Clin Chem (2002) 48(8):1212-7]. The mixture was centrifuged at 11,900×g for 15 minutes at 4° C. and the aqueous layer was transferred into new tubes. One volume of 70% ethanol was added to one volume of the aqueous layer. The mixture was then transferred to an RNeasy minicolumn (RNeasy mini kit, Qiagen, Valencia, Calif.) following the manufacturer's recommendations. On-column DNase treatment was carried out to remove any contaminating DNA (RNase-Free DNase Set, Qiagen, Valencia, Calif.). Total RNA was eluted in 30 μl RNase-free water and stored at −80° C.

Real-Time Quantitative RT-PCR:

Amplification of specific cell free mRNA was conducted using a one-step real-time quantitative RT-PCR with specific primers directed at each gene of interest. RT-PCR primers were intron spanning, to reduce DNA contamination, and a NCBI-blast check was done to rule out non-specific amplifications.

RT-PCRs were set up according to the manufacturer's instructions (EZ rTth RNA PCR reagent set, Applied Biosystems, Foster City, Calif.) in a reaction volume of 20 μl. In detail, 5 μl extracted plasma RNA was amplified using 100-500 nM PCR primers. Each sample was analyzed in duplicates and the corresponding calibration curves were processed simultaneously in triplicates for each analysis. No template control (NTC) was also included in every analysis.

The RT-PCR thermal profile used in accordance with the present invention was as follows: The reaction was initiated at 50° C. for 20 minutes reverse transcription and a 5 minute denaturation at 95° C. Next, 50 cycles of PCR were carried out as follows: 15 seconds of denaturation at 95° C. followed by 30 seconds of annealing/extension at 60° C.

Blood Collection from Patients Suffering from Myocardial Infarction:

For analyzing the level of cell free RNA in patients suffering from myocardial infarction (MI), an assay kit from Applied Biosystems was used (Assays-on-Demand™, Table 3). The assay uses a collection of pre-designed primer and probe sets for quantitative real-time PCR gene expression studies.

RNA was extracted by magnetic beads (Magmax kit, by Applied Biosystems; AM1836) and used in a one step RT-PCR kit (QuantiFast Probe RT-PCR Plus Kit from QIAGEN; 204482). Quantification of RNA was carried out in accordance with a relative quantification method as explained below.

### Example 1

### p21 mRNA is Elevated in the Serum of Complicated Hypoxic Pregnancies as Compared to Normal Pregnancies

### Results

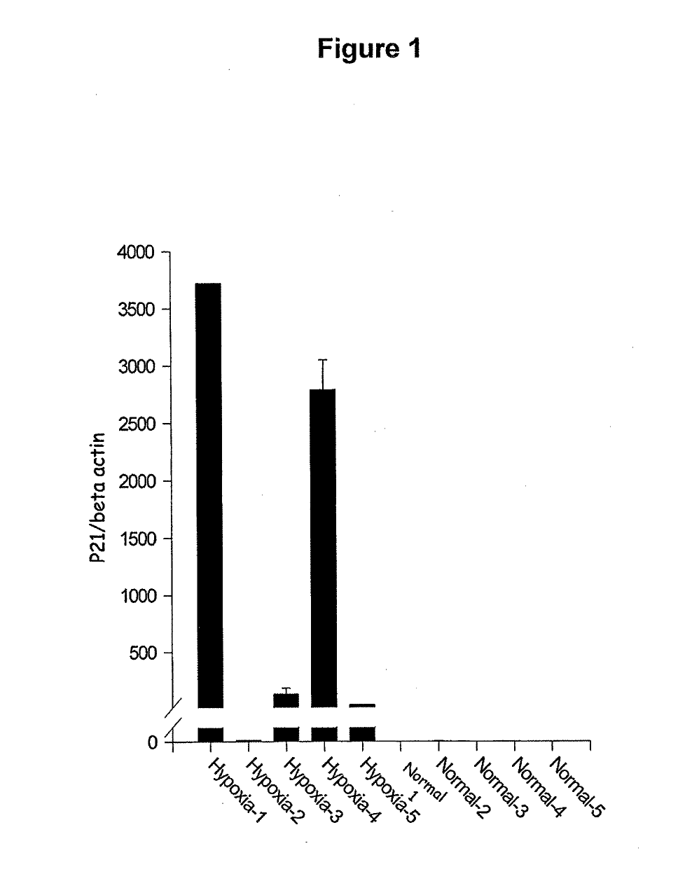

Results indicated that p21 gene expression is highly elevated in complicated hypoxic pregnancies (FIG. 1). Positive p21 gene expression was observed in four out of the five RNA samples obtained from subjects with complicated pregnancies, whereas no p21 gene expression was observed in the five samples obtained from subjects with normal pregnancies.

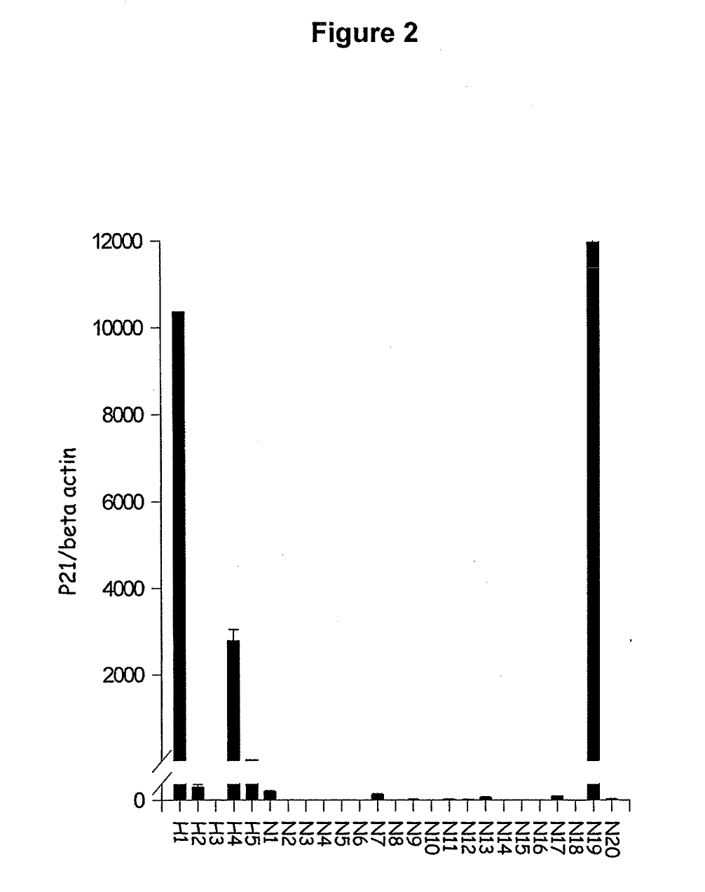

In a further study, p21 gene expression was tested in 20 RNA samples of normal pregnancies and results were compared to the elevated p21 gene expression observed in complicated pregnancies (as depicted FIG. 1). As illustrated in FIG. 2, p21 gene expression was highly elevated in only one out of the twenty cases of normal pregnancies tested (case number 19) while a minor elevation was detected in four cases (cases number 1, 7, 13 and 17).

Altogether the present results substantiate circulating p21 mRNA as a non-invasive marker for predicting fetal stress.

### Example 2

Up-Regulation in RNA Expression of p21, MDM2 and HIF1α in Plasma Samples of Women with Complicated Hypoxic Pregnancies as Determined in a Broad Clinical Study

### Results

Following the initial results (as depicted in Example 1), the study was extended to a larger group of subjects (54 subjects in total), of these, 31 normal pregnancy subjects and 23 complicated pregnancy subjects. Maternal plasma RNA samples were tested for gene expression of genes associated with hypoxia and stress, specifically, p21 (GeneBank Accession No. U09579), VEGF (GeneBank Accession No. NM_{—}001025366), MDM2 (GeneBank Accession No. M92424), HIF1α (GeneBank Accession No. NM_{—}001530.2) and TP53I3 (GeneBank Accession No. NM_{—}147184.1) using primers and probes as shown in Table 2.

As depicted in Table 4A below, out of 23 hypoxic pregnancy subjects tested 13 were tested positive for p21 gene expression and 10 were recorded negative for p21 gene expression. Furthermore, out of 31 normal pregnancy subjects tested only 2 were tested positive for p21 gene expression and 29 were recorded negative for p21 gene expression. Chi-square test results of p21 gene expression in hypoxic pregnancies versus normal pregnancies depicted p was smaller that 0.001 (Table 4B).

TABLE 4A
p21 gene expression
H/N * p21 Crosstabulation

p21

| | | | − | + | Total |
|---|---|---|---|---|---|
| H/N | H | Count | 10 | 13 | 23 |
| | | % within H/N | 43.5% | 56.5% | 100.0% |
| | | % within p21 | 25.6% | 86.7% | 42.6% |
| | N | Count | 29 | 2 | 31 |
| | | % within H/N | 93.5% | 6.5% | 100.0% |
| | | % within p21 | 74.4% | 13.3% | 57.4% |
| Total | | Count | 39 | 15 | 54 |
| | | % within H/N | 72.2% | 27.8% | 100.0% |
| | | % within p21 | 100.0% | 100.0% | 100.0% |

TABLE 4B
Chi-square test
Chi-Square Tests

| | | | Asymp. Sig. | Exact Sig. | Exact Sig. |
|---|---|---|---|---|---|
| | Value | df | (2-sided) | (2-sided) | (1-sided) |
| Pearson Chi-Square | 16.500^{b} | 1 | .000 | | |
| Continuity Correc- | 14.099 | 1 | .000 | | |
| tion^{a} | | | | | |
| Likelihood Ratio | 17.487 | 1 | .000 | | |
| Fisher's Exact Test | | | | .000 | .000 |
| N of Valid Cases | 54 | | | | |

^{a}Computed only for a 2 × 2 table
^{b}0 cells (.0%) have expected count less than 5. The minimum expected count is 6.39.

As depicted in Table 5A below, out of 23 hypoxic pregnancy subjects tested 10 were tested positive for VEGF gene expression and 13 were recorded negative for VEGF gene expression. Furthermore, out of 31 normal pregnancy subjects tested 6 were tested positive for VEGF gene expression and 25 were recorded negative for VEGF gene expression. Chi-square test results of VEGF gene expression in hypoxic pregnancies versus normal pregnancies depicted p=0.053 (Table 5B).

TABLE 5A
VEGF gene expression
H/N * vegf Crosstabulation

vegf

| | | | − | + | Total |
|---|---|---|---|---|---|
| H/N | H | Count | 13 | 10 | 23 |
| | | % within H/N | 56.5% | 43.5% | 100.0% |
| | | % within vegf | 34.2% | 62.5% | 42.6% |
| | N | Count | 25 | 6 | 31 |
| | | % within H/N | 80.6% | 19.4% | 100.0% |
| | | % within vegf | 65.8% | 37.5% | 57.4% |
| Total | | Count | 38 | 16 | 54 |
| | | % within H/N | 70.4% | 29.6% | 100.0% |
| | | % within vegf | 100.0% | 100.0% | 100.0% |

TABLE 5B
Chi-square test
Chi-Square Tests

| | | | Asymp. Sig. | Exact Sig. | Exact Sig. |
|---|---|---|---|---|---|
| | Value | df | (2-sided) | (2-sided) | (1-sided) |
| Pearson Chi-Square | 3.685^{b} | 1 | .055 | | |
| Continuity Correc- | 2.619 | 1 | .106 | | |
| tion^{a} | | | | | |
| Likelihood Ratio | 3.676 | 1 | .055 | | |
| Fisher's Exact Test | | | | .074 | .053 |
| N of Valid Cases | 54 | | | | |

^{a}Computed only for a 2 × 2 table
^{b}0 cells (.0%) have expected count less than 5. The minimum expected count is 6.81.

As depicted in Table 6A below, out of 22 hypoxic pregnancy subjects tested 12 were tested positive for MDM2 gene expression and 10 were recorded negative for MDM2 gene expression. Furthermore, out of 31 normal pregnancy subjects tested only were tested positive for MDM2 gene expression and 26 were recorded negative for MDM2 gene expression. Chi-square test results of MDM2 gene expression in hypoxic pregnancies versus normal pregnancies depicted p=0.004 (Table 6B).

TABLE 6A
MDM2 gene expression
H/N * mdm2 Crosstabulation

| | | | mdm2 | | |
|---|---|---|---|---|---|
| | | | − | + | Total |
| H/N | H | Count | 10 | 12 | 22 |
| | | % within H/N | 45.5% | 54.5% | 100.0% |
| | | % within mdm2 | 27.8% | 70.6% | 41.5% |
| | N | Count | 26 | 5 | 31 |
| | | % within H/N | 83.9% | 16.1% | 100.0% |
| | | % within mdm2 | 72.2% | 29.4% | 58.5% |
| Total | | Count | 36 | 17 | 53 |
| | | % within H/N | 67.9% | 32.1% | 100.0% |
| | | % within mdm2 | 100.0% | 100.0% | 100.0% |

TABLE 6B
Chi-square test
Chi-Square Tests

| | | | Asymp. Sig. | Exact Sig. | Exact Sig. |
|---|---|---|---|---|---|
| | Value | df | (2-sided) | (2-sided) | (1-sided) |
| Pearson Chi-Square | 8.717^{b} | 1 | .003 | | |
| Continuity Correc- | 7.042 | 1 | .008 | | |
| tion^{a} | | | | | |
| Likelihood Ratio | 8.800 | 1 | .003 | | |
| Fisher's Exact Test | | | | .006 | .004 |
| N of Valid Cases | 53 | | | | |

^{a}Computed only for a 2 × 2 table
^{b}0 cells (.0%) have expected count less than 5. The minimum expected count is 7.06.

As depicted in Table 7A below, out of 22 hypoxic pregnancy subjects tested 10 were tested positive for HIF1α gene expression and 12 were recorded negative for HIF1α gene expression. Furthermore, out of 31 normal pregnancy subjects tested 0 were tested positive for HIF1α gene expression and 31 were recorded negative for HIF1α gene expression. Chi-square test results of HIF1α gene expression in hypoxic pregnancies versus normal pregnancies depicted p was smaller that 0.001 (Table 7B).

TABLE 7A
HIF1α gene expression
H/N * hif1a Crosstabulation

hif1a

| | | | − | + | Total |
|---|---|---|---|---|---|
| H/N | H | Count | 12 | 10 | 22 |
| | | % within H/N | 54.5% | 45.5% | 100.0% |
| | | % within hif1a | 27.9% | 100.0% | 41.5% |
| | N | Count | 31 | 0 | 31 |
| | | % within H/N | 100.0% | .0% | 100.0% |
| | | % within hif1a | 72.1% | .0% | 58.5% |
| Total | | Count | 43 | 10 | 53 |
| | | % within H/N | 81.1% | 18.9% | 100.0% |
| | | % within hif1a | 100.0% | 100.0% | 100.0% |

TABLE 7B
Chi-square test
Chi-Square Tests

| | | | Asymp. Sig. | Exact Sig. | Exact Sig. |
|---|---|---|---|---|---|
| | Value | df | (2-sided) | (2-sided) | (1-sided) |
| Pearson Chi-Square | 17.368^{b} | 1 | .000 | | |
| Continuity Correc- | 14.525 | 1 | .000 | | |
| tion^{a} | | | | | |
| Likelihood Ratio | 21.020 | 1 | .000 | | |
| Fisher's Exact Test | | | | .000 | .000 |
| N of Valid Cases | 53 | | | | |

^{a}Computed only for a 2 × 2 table
^{b}1 cells (25.0%) have expected count less than 5. The minimum expected count is 4.15.

As depicted in Table 8A below, out of 22 hypoxic pregnancy subjects tested 6 were tested positive for TP53I3 gene expression and 16 were recorded negative for TP53I3 gene expression. Furthermore, out of 23 normal pregnancy subjects tested 5 were tested positive for TP53I3 gene expression and 18 were recorded negative for TP53I3 gene expression. Chi-square test results of TP53I3 gene expression in hypoxic pregnancies versus normal pregnancies depicted p=0.466 (Table 8B).

TABLE 8A
TP53I3 gene expression
H/N * TP5313 Crosstabulation

| | | | TP5313 | | |
|---|---|---|---|---|---|
| | | | − | + | Total |
| H/N | H | Count | 16 | 6 | 22 |
| | | % within H/N | 72.7% | 27.3% | 100.0% |
| | | % within TP5313 | 47.1% | 54.5% | 48.9% |
| | N | Count | 18 | 5 | 23 |
| | | % within H/N | 78.3% | 21.7% | 100.0% |
| | | % within TP5313 | 52.9% | 45.5% | 51.1% |
| Total | | Count | 34 | 11 | 45 |
| | | % within H/N | 75.6% | 24.4% | 100.0% |
| | | % within TP5313 | 100.0% | 100.0% | 100.0% |

TABLE 8B
Chi-square test
Chi-Square Tests

| | | | Asymp. Sig. | Exact Sig. | Exact Sig. |
|---|---|---|---|---|---|
| | Value | df | (2-sided) | (2-sided) | (1-sided) |
| Pearson Chi-Square | .186^{b} | 1 | .666 | | |
| Continuity Correc- | .007 | 1 | .932 | | |
| tion^{a} | | | | | |
| Likelihood Ratio | .187 | 1 | .666 | | |
| Fisher's Exact Test | | | | .738 | .466 |
| N of Valid Cases | 45 | | | | |

^{a}Computed only for a 2 × 2 table
^{b}0 cells (.0%) have expected count less than 5. The minimum expected count is 5.38.

As illustrated in Table 9 below, correlating p21, MDM2 and HIF1α gene expressions in hypoxic pregnancies compared to normal pregnancies revealed that only 4 hypoxic pregnancy subjects were negative for expression of these 3 genes, whereas 24 normal pregnancy subjects were negative for expression of these 3 genes. Furthermore, 5 hypoxic pregnancy subjects were positive for expression of p21, MDM2 and HIF1α, whereas none of the normal pregnancy subjects were recorded positive.

TABLE 9
relationship between p21, MDM2 and HIF1α gene expressions
p21 * mdm2 * hif1a * H/N Crosstabulation
Count

mdm2

| H/N | hif1a | | | − | + | Total |
|---|---|---|---|---|---|---|
| H | − | p21 | − | 4 | 2 | 6 |
| | | | + | 3 | 3 | 6 |
| | | Total | | 7 | 5 | 12 |
| | + | p21 | − | 1 | 2 | 3 |
| | | | + | 1 | 5 | 6 |
| | | Total | | 2 | 7 | 9 |
| N | − | p21 | − | 24 | 5 | 29 |
| | | | + | 2 | 0 | 2 |
| | | Total | | 26 | 5 | 31 |

Similarly, as illustrated in Table 10 below, correlating p21, VEGF and HIF1α gene expressions in hypoxic pregnancies compared to normal pregnancies revealed that only 5 hypoxic pregnancy subjects were negative for expression of these 3 genes, whereas 23 normal pregnancy subjects were negative for expression of these 3 genes. Furthermore, 3 hypoxic pregnancy subjects were positive for expression of p21, VEGF and HIF1α, whereas none of the normal pregnancy subjects were recorded positive.

TABLE 10
relationship between p21, VEGF and HIF1α H/N gene expressions
p21 * vegf * hif1a * H/N Crosstabulation
Count

vegf

| H/N | hif1a | | | − | + | Total |
|---|---|---|---|---|---|---|
| H | − | p21 | − | 5 | 1 | 6 |
| | | | + | 3 | 3 | 6 |
| | | Total | | 8 | 4 | 12 |
| | + | p21 | − | 1 | 3 | 4 |
| | | | + | 3 | 3 | 6 |
| | | Total | | 4 | 6 | 10 |
| N | − | p21 | − | 23 | 6 | 29 |
| | | | + | 2 | 0 | 2 |
| | | Total | | 25 | 6 | 31 |

Summarizing the gene expression of the different hypoxia related genes in hypoxic pregnancies (Tables 11 and 12, below) revealed that p21 gene expression and/or HIF1α gene expression are the most notably upregulated genes in hypoxic pregnancies. Thus, expressions of both these genes, together or separately, among other p53 inducible genes that are over expressed or repressed in conditions associated with hypoxia, mark hypoxic stress during pregnancy.

TABLE 11
Model if Term Removed

| | | | Change | | |
|---|---|---|---|---|---|
| | | Model Log | in −2 Log | | Sig. of the |
| Variable | | Likelihood | Likelihood | df | Change |
| Step | p21 | −25.007 | 9.203 | 1 | .002 |
| 1 | vegf | −20.534 | .255 | 1 | .613 |
| | hif1a | −26.563 | 12.313 | 1 | .000 |
| Step | p21 | −25.459 | 9.851 | 1 | .002 |
| 2 | hif1a | −27.943 | 14.819 | 1 | .000 |

TABLE 12
Model if Term Removed

| | | | Change | | |
|---|---|---|---|---|---|
| | | Model Log | in −2 Log | | Sig. of the |
| Variable | | Likelihood | Likelihood | df | Change |
| Step | p21 | −22.518 | 9.008 | 1 | .003 |
| 1 | ercc5 | −18.054 | .081 | 1 | .776 |
| | mdm2 | −18.346 | .664 | 1 | .415 |
| | vegf | −18.279 | .530 | 1 | .466 |
| | hif1a | −21.930 | 7.832 | 1 | .005 |
| Step | p21 | −22.558 | 9.009 | 1 | .003 |
| 2 | mdm2 | −18.459 | .810 | 1 | .368 |
| | vegf | −18.330 | .553 | 1 | .457 |
| | hif1a | −21.966 | 7.825 | 1 | .005 |
| Step | p21 | −23.236 | 9.810 | 1 | .002 |
| 3 | mdm2 | −18.696 | .732 | 1 | .392 |
| | hif1a | −23.246 | 9.832 | 1 | .002 |
| Step | p21 | −24.152 | 10.911 | 1 | .001 |
| 4 | hif1a | −25.058 | 12.725 | 1 | .000 |

### Example 3

### p53-Induced Gene Activation in Patients Suffering from Acute Myocardial Infarction

### Study Subjects:

A group of 100 subjects participated in the study. Fifty subjects were patients having acute myocardial infarction (MI) hospitalized in the cardiac intensive care unit in Meir Hospital (Kfar Saba). All patients were men over 30 years old. Fifty subjects were patients undergoing non-invasive ischemic assessment which did not show an acute ischemia (the control group).

### MI Patients:

The MI patients suffered from acute MI accompanied by an ST segment elevation. The patients were hospitalized due to continuous chest pain that was reported to have occurred for at least 1 hour but no more than 6 hours. All MI patients were intended for urgent catheterization and demonstrated at least one of the following:
1. elevated ST in anterior wall
2. new Left bundle branch block (LBBB)
3. ST elevation in posterior wall with evidence of an electrocardiographic involvement of a lateral or posterior wall

### Control Group:

The control group consisted of 50 patients undergoing non-invasive ischemic assessment using echocardiogram or an echocardiogram stress test which did not show an acute ischemia.

Blood samples were analyzed for the expression of the genes BNIP3L, P21, MDM2, HIF1α, NOTCH1, BTG2, TGFβ3, ZMAT3 and ERCC5. RNA was isolated from the patients and amplified. Quantification of cell free RNA in blood samples of patients was carried out in accordance with a relative quantification method (Livak K J and Schmittgen T D. Methods 2001; 25(4):402-8; Marisa L W and Juan F M, BioTechniques 2005; 39:75-85) which determines the changes in steady-state mRNA levels of a gene across multiple samples and expresses it relative to the levels of an internal control RNA. Total RNA extracted from A2780 (human ovarian cancer cell line) was used as external standard (calibrator) for relative quantification. The fold increase of the test samples as compared to the calibrator (i.e. RNA from the line A2780) was quantified. All the patient's RNA samples (after normalization to an internal control being GAPDH) were compared to the level of expression in the A2780 cell line.

### For the MI Group:

Two blood samples were taken:
1. Upon arrival at the hospital (between the arrival time to the emergency unit and until catheterization), t=0;
2. Four hours after catheterization; t=4.

### for the Control Group:

One blood sample was taken before performing the assay (echocardiogram or echocardiogram stress test).

### Statistical Analysis:

1. A parametric t-test was conducted for cell free RNA levels at t=0 (in MI patient's) vs. control; t=4 (in MI patient's) vs. control and t=0 vs. t=4 (Table 13, results depict p-value; * p<0.05, ** p<0.01).

TABLE 13
t-test

| Gene | t = 0/control | t = 4/control | t = 0/t = 4 |
|---|---|---|---|
| BNIP3L | 0.3 | 0.37 | 0.16 |
| P21 | 0.0012** | 0.001** | 0.21 |
| MDM2 | 0.02* | 0.11 | 0.08 |
| HIF1-α | 0.0002*** | 0.00003 | 0.4 |
| NOTCH1 | 0.18 | 0.14 | 0.19 |
| BTG2 | 0.073 | 0.12 | 0.00004*** |
| TGF-β3 | N/A | N/A | N/A |
| ZMAT3 | N/A | N/A | N/A |
| ERCC5 | 0.07 | 0.06 | 0.4 |

2. A Mann Whitney non-parametric statistical test (Wilcoxon rank-sum test) was carried out in cases where cell free RNA was not detected at t=0 and/or t=4 and/or in the control in some of the blood samples (Table 14, results depict p-value; * p<0.05, ** p<0.01, *** p<0.001)

TABLE 14
Mann-Whitney test

| Gene | t = 0/control | t = 4/control | t = 0/t = 4 |
|---|---|---|---|
| BNIP3L | N/A | N/A | N/A |
| P21 | 0.0007*** | 0.28 | 0.0017** |
| MDM2 | 0.14 | 0.5 | 0.08 |
| HIF1-α | 0.017* | 0.4 | 0.017* |
| NOTCH1 | 0.0024** | 0.14 | 0.0034** |
| BTG2 | 0.0002*** | 0.24 | 0.0001*** |
| TGF-β3 | 0.15 | 0.26 | 0.013* |
| ZMAT3 | 0.094 | 0.036* | 0.16 |
| ERCC5 | 0.11 | 0.44 | 0.08 |

The results reveal that the expression of the p21, MDM2, HIF-1α, NOTCH1, BTG2 genes is indicative of acute MI (Tables 13-14, left column). The genes p21, HIF-1α, NOTCH1, TGF-β3 and BTG2 were shown to be predictive markers for the success of treatment of MI (Tables 13-14, right column).

The genes p21, MDM2, HIF 1-α, NOTCH 1 and BTG2 were shown to be predictive markers for the return of gene expression to normal values, as depicted in the t=4 vs. control (middle columns in tables 13, 14).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the present invention.

1-23. (canceled)

24. A method for detecting a condition associated with hypoxia in a subject, the method comprising determining in a biological sample obtained from the subject the level of a cell free Ribonucleic acid (RNA) of at least one p53 inducible gene, wherein a level of the cell free RNA above or below a predetermined range associated with the at least one p53 inducible gene, is indicative that the subject has a condition associated with hypoxia.

25. The method of claim 24, wherein said predetermined range correlates with severity of the condition associated with hypoxia, and said level of the cell free RNA of at least one p53 inducible gene is compared with said predetermined range, the comparison allowing determination of the severity of the a condition associated with hypoxia in the subject.

26. A method for determining the effectiveness of a therapeutic treatment of a condition associated with hypoxia in a subject comprising determining the level of cell free RNA of at least one p53 inducible gene from two or more biological samples obtained from the subject at two or more time points, at least one of the time points is during or after the treatment, wherein:
(i) for a p53 inducible gene that is over-expressed in a condition associated with hypoxia a decrease in the level of the cell free RNA of the p53 inducible gene between the two or more samples being indicative of effectiveness of the therapeutic treatment;
(ii) for a p53 inducible gene that is repressed in a condition associated with hypoxia an increase in the level of the cell free RNA of the p53 inducible gene between the two or more samples being indicative of effectiveness of the therapeutic treatment.

27. A method for selecting a subject suffering from a condition associated with hypoxia, to receive therapeutic treatment to treat the condition, the method comprising determining the level of cell free RNA of at least one p53 inducible gene in a biological sample obtained from the subject and selecting the subject to receive said therapeutic treatment if the level of cell free RNA of at least one p53 inducible gene is above or below a predetermined range associated with the at least one p53 inducible gene.

28. The method of claim 24, wherein said sample is a bodily fluid sample.

29. The method of claim 26, wherein said sample is a bodily fluid sample.

30. The method of claim 27, wherein said sample is a bodily fluid sample.

31. The method of claim 28, wherein said bodily fluid sample is a blood sample.

32. The method of claim 31, wherein said blood sample is selected from the group consisting of a serum sample and a plasma sample.

33. The method of claim 29, wherein said bodily fluid sample is a blood sample.

34. The method of claim 33, wherein said blood sample is selected from the group consisting of a serum sample and a plasma sample.

35. The method of claim 30, wherein said bodily fluid sample is a blood sample.

36. The method of claim 35, wherein said blood sample is selected from the group consisting of a serum sample and a plasma sample.

37. The method of claim 24, wherein level of cell free RNA of at least one p53 inducible gene is determined by RT-PCR or by real-time quantitative RT-PCR.

38. The method of claim 26, wherein level of cell free RNA of at least one p53 inducible gene is determined by RT-PCR or by real-time quantitative RT-PCR.

39. The method of claim 27, wherein level of cell free RNA of at least one p53 inducible gene is determined by RT-PCR or by real-time quantitative RT-PCR.

40. The method of claim 24, wherein the condition associated with hypoxia is selected from cardiovascular diseases, cancer, cerebrovascular accident (CVA) and fetal stress.

41. The method of claim 26, wherein the condition associated with hypoxia is selected from cardiovascular diseases, cancer, cerebrovascular accident (CVA) and fetal stress.

42. The method of claim 27, wherein the condition associated with hypoxia is selected from cardiovascular diseases, cancer, cerebrovascular accident (CVA) and fetal stress.

43. The method claim 24, wherein the at least one p53 inducible gene is selected from p21 (GeneBank Accession No. Nm_{—}000389), BTG2 (GeneBank Accession No. Nm_{—}006763), HIF-1α (GeneBank Accession No. Nm_{—}001530), NOTCH1 (GeneBank Accession No. Nm_{—}017617), TGFβ3 (GeneBank Accession No. Nm_{—}003239) and ZMAT3 (GeneBank Accession No. Nm_{—}0022470).

44. The method claim 26, wherein the at least one p53 inducible gene is selected from p21 (GeneBank Accession No. Nm_{—}000389), BTG2 (GeneBank Accession No. Nm_{—}006763), HIF-1α (GeneBank Accession No. Nm_{—}001530), NOTCH1 (GeneBank Accession No. Nm_{—}017617), TGFβ3 (GeneBank Accession No. Nm_{—}003239) and ZMAT3 (GeneBank Accession No. Nm_{—}0022470).

45. The method claim 27, wherein the at least one p53 inducible gene is selected from p21 (GeneBank Accession No. Nm_{—}000389), BTG2 (GeneBank Accession No. Nm_{—}006763), HIF-1α (GeneBank Accession No. Nm_{—}001530), NOTCH1 (GeneBank Accession No. Nm_{—}017617), TGFβ3 (GeneBank Accession No. Nm_{—}003239) and ZMAT3 (GeneBank Accession No. Nm_{—}0022470).
